(19) United States
(12) Patent Application Publication
(10) Pub. No.: US 2007/0060615 A1
(43) Pub. Date: Mar. 15, 2007

(54) Method of Modulating Apoptosis Through Modulation of E2F1

(75) Inventors: William Douglas Cress JR., Lutz, FL (US); Eric B. Haura, Tampa, FL (US); Xihong Ma, Tampa, FL (US)

Correspondence Address: SMITH HOPEN, PA, 180 PINE AVENUE NORTH, OLDSMAR, FL, 34677, US

(73) Assignee: University of South Florida, Tampa, FL (US)

(21) Appl. No.: 11/534,459

(22) Filed: Sep. 22, 2006

Related U.S. Application Data
(62) Division of application No. 10/708,239, filed on Feb. 18, 2004.
(60) Provisional application No. 60/319,955, filed on Feb. 18, 2003.

(51) Int. Cl. A61K 31/452

(52) U.S. Cl. 514/320

(57) ABSTRACT

Provided is a method and vector for modulating apoptosis in a target cell population. Flavopiridol treatment leads to apoptosis via a mechanism associated with downregulation of Mcl-1. E2F1 leads to transcriptional repression of Mcl-1 and subsequently apoptosis. Given the ability of cyclin/cyclin-dependent kinase 2 antagonists to kill transformed cells, flavopiridol may stabilize E2F1 and enhance apoptosis via repression of Mcl-1. Flavopiridol is associated with a dose-dependent increase in E2F1 protein levels, a corresponding reduction in Mcl-1, and apoptosis in lung carcinoma cells. Treatment of cells with 200 nM flavopiridol results in the rapid elevation of E2F1 and reduction in Mcl-1 levels within 12 hours of treatment. The elevation of E2F1 and reduction in Mcl-1 clearly precedes the induction of apoptosis. Cell lines that constitutively express Mcl-1 under the control of the cytomegalovirus promoter have no reductions in Mcl-1 levels with flavopiridol treatment and are resistant to apoptosis induced by flavopiridol.

### CROSS REFERENCE TO RELATED APPLICATIONS

This application is a divisional of prior application Ser. No. 10/708,239, entitled “E2F1 Inhibitor to Prevent Apoptosis”, filed on Feb. 18, 2004, the contents of which are incorporated herein by reference, which claims the benefit of U.S. Provisional Patent Application Ser. No. 60/319,955, entitled, “E2F1 Inhibitor to Prevent Apoptosis”, filed Feb. 18, 2003, the contents of which are herein incorporated by reference.

### STATEMENT OF GOVERNMENT INTEREST

This invention was made with Government support under Grant No. R29CA78214 awarded by the National Institute of Health. The Government has certain rights in the invention.

### BACKGROUND OF THE INVENTION

Considerable evidence suggests that deregulation of the Rb 3/E2F pathway is a hallmark of human cancers. Regulation of this pathway is centrally controlled through the action of the G1 cyclins (D, E, and A) in conjunction with the catalytic partner cdks, such as cdk2 and cdk4. A general paradigm exists in which growth stimuli lead to enhanced levels of cyclin D/cdk4 and cyclin E/cdk2 levels, which lead to phosphorylation of Rb and enhanced activity of the E2F family of transcription factors. The resulting increase in E2F activity leads to the induction of genes important in S phase, such as dihydrofolate reductase, thymidine kinase, and DNA polymerase-α. Once cells are in S phase, E2F activity is no longer necessary, and the subsequent rise in cyclin A/cdk2 activity (induced by E2F1) leads to down-regulation of E2F activity and the cessation of S phase. This downregulation of E2F activity by cyclin A is required for orderly S-phase progression, and in its absence, apoptosis occurs. Therefore, E2F activity is negatively regulated by Rb in G 0 and by cyclin A/cdk2 in S phase.

This Rb-E2F pathway can be corrupted by multiple mechanisms, including amplification of the G 1 cyclins, loss of the cdk inhibitors such as p161NK4a or p27, or direct mutations of Rb. In some cancer cell lines, overexpression of cdk inhibitors such as p16 or p27 can cause cell cycle arrest implying that the pharmacological inhibition of cdk activity may be a rational therapeutic target for cancers. As a result, attempts have been made to develop therapeutic agents that specifically target the cdks and arrest tumor growth. One such candidate is flavopiridol, a semisynthetic flavonoid, which was found to inhibit cell growth in both the G 1 and G 2-M phases of the cell cycle. Flavopiridol directly inhibits the cdks-1, -2, -4, -6, and -7 by competition with ATP with IC 5 0 concentrations of ˜0.1 μM. Although originally thought of as a cytostatic agent by virtue of its ability to inhibit cdk activity and lead to cell cycle arrest, flavopiridol may down-regulate the antiapoptotic proteins Mcl-1 and XIAP and lead to apoptosis.

The mechanism for flavopiridol-induced apoptosis in cancer cells is through the activation of E2F1 activity and repression of Mcl-1. [C]dk2 antagonists can potentiate the apoptotic function of E2F1, possibly by preventing the negative regulation of E2F1 activity by cyclin A/cdk2 activity. The first member of the E2F family identified, E2F1, has the unique ability to induce genes important for not only S-phase initiation but also apoptosis or programmed cell death. The best characterized pathway for E2F1-mediated cell death consists of the transcriptional regulation of p14 ARF, which binds and negatively regulates MDM2, which subsequently leads to elevated levels of p53. However, E2F1 may lead to apoptosis independent of the p14ARF-MDM2-p53 pathway. E2F1 directly regulates members of the Bcl-2 family and leads to apoptosis. E2F1 leads to transcriptional repression of the Mcl-1 gene, and subsequent falls in Mcl-1 protein levels drives cells into apoptosis. Flavopiridol induces apoptosis through elevations of E2F1 activity (through the down-regulation of cyclin A/cdk2 activity) and subsequent down-regulation of Mcl-1 expression, which finally culminates in apoptosis; as evidenced by the apoptotic function of flavopiridol which is associated with downregulation of Mcl-1. The treatment of cells with flavopiridol results in parallel increases in E2F1 levels and decreases in Mcl-1 levels that precede the induction of apoptosis. Cells that are deficient in E2F1 have reduced apoptosis with treatment with flavopiridol, and cells constitutively expressing Mcl-1 are resistant to apoptosis induced by flavopiridol.

### SUMMARY OF INVENTION

Provided is a method and vector for modulating apoptosis in a target cell population. The inventive method of modulating apoptosis in a target cell population comprises the step of regulating the expression of E2F1 whereby the expression of Mcl-1 is increased responsive to the downregulation of E2F1. The method is useful when the apoptosis being modulated is Flavopiridol-induced apoptosis. The occurrence of apoptosis is reduced by repression of E2F1. In one embodiment, the repression of E2F1 is accomplished by contacting a target cell with an RNA inhibitor molecule, wherein the RNA inhibitor molecule is a BS/U6 E2F 1 RNAi plasmid.

The inventive vector for modulating the expression of E2F1 in a target cell population comprises a BS/U6 E2F1 RNAi plasmid. The BS/U6 E2F1 RNAi plasmid further comprises a parent plasmid, a first nucleotide sequence, wherein the first nucleotide sequence is SEQ. NO. 1, a second nucleotide sequence, wherein the second nucleotide sequence is SEQ. NO. 2, a third nucleotide sequence, wherein the third nucleotide sequence is SEQ. NO. 3, and a fourth nucleotide sequence, wherein the fourth nucleotide sequence is SEQ. NO. 4. The parent plasmid is an empty BS/U6 RNAi vector further comprising an ApaI binding site, a HindIII binding site, and an EcoRI binding site. In the first step of the construction, E2F1 RNAi oligo 1a (SEQ. NO. 1) and 1b (SEQ. NO. 2) were annealed and ligated into the Apa I (made blunt with T4 DNA polymerase) and Hin dIII sites of BS/U6. In the second step, E2F1 RNAi oligo 1c (SEQ. NO. 3) and 1d (SEQ. NO.4) were annealed and ligated into the Hin dIII and Eco RI sites of the intermediate vector. The final vector expresses a single-stranded RNA from the U6 promoter, which forms a 21-bp double-stranded stem, a 6-bp loop, and, at the 3′-end, a short stretch of thymidine residues corresponding to the termination sequence of RNA polymerase III. The sequence of oligo 1a (SEQ. NO. 1) is 5′-GGGGGAGAAGTCACGCTATGA-3′, oligo 1b (SEQ. NO. 2) is 5′-GCTTCATAGCGTGACTTCTCCCCC-3′, oligo 1c (SEQ. NO. 3) is 5′-GCTTCATAGCGTGACTTCTCCCCCTTTTTG-3′, and oligo 1d (SEQ. NO. 4) is 5′-ATTCAAAAAGGGGGAGAAGTCACGCTATGA-3′. To determine whether the E2F1 RNAi was functional, it was cotransfected together with a pcDNA3-E2F1 expression vector. The BS/U6 E2F1 RNAi vector abolished expression of E2F1 from the vector as measured by both E2F1 western and by a luciferase assay, which measured E2F1 transcriptional activation.

### BRIEF DESCRIPTION OF THE DRAWINGS

For a fuller understanding of the invention, reference should be made to the following detailed description, taken in connection with the accompanying drawings, in which:

FIG. 1 Flavopiridol induces a dose-dependent increase in E2F1, a parallel decrease in Mcl-1, and apoptosis. (A), H1299 lung carcinoma cells were treated with increasing doses of flavopiridol as indicated for 48 hours, and whole cell protein extracts were prepared for Western blotting with antibodies for E2F1, Mcl-1, and actin to control for protein loading. (B) Parallel treated cells were harvested for determination of apoptosis measured by incorporation of Apo-BrdUrd at the times indicated. Percentage of cells corresponds to the percentage of cells labeling with Apo-BrdUrd and is plotted at indicated time intervals.

FIG. 2 Flavopiridol induces a time-dependent rise in E2F1 levels and fall in Mcl-1 levels that precedes the onset of apoptosis. (A) H1299 cells were treated with 200 nM flavopiridol for the times indicated, and whole cell extracts were probed with antibodies specific for E2F1, Mcl-1, and actin to control for protein loading. (B) Parallel treated cells were harvested for determination of apoptosis measured by incorporation of Apo-BrdUrd at the times indicated. Percentage of cells corresponds to the percentage of cells labeling with Apo-BrdUrd and is plotted at indicated time intervals.

FIG. 3 Reduction in Mcl-1 and elevations in E2F1 precedes apoptosis induced by flavopiridol. (A) H1299 cells were treated with 200 nM flavopiridol for the times indicated, and whole cell extracts were probed with antibodies specific for E2F1, Rb, and Mcl-1. Flavopiridol treatment results in a sustained elevation in E2F1 levels until 72 hours and parallel reduction in Mcl-1 protein levels. No significant changes in total Rb levels or Rb phosphorylation were noted. Percentage of cells in G1-G1, S phase, and G2-M are shown at the indicated times after flavopiridol treatment. (B) Parallel treated cells were harvested for determination of apoptosis measured by incorporation of Apo-BrdUrd at the times indicated. Percentage of cells corresponds to the percentage of cells labeling with Apo-BrdUrd.

FIG. 4 H1299 lung cancer cells, constitutively expressing human Mcl-1, are resistant to flavopiridol-induced apoptosis. (A) H1299 cells, stably expressing human Mcl-1, and control cells were harvested for total protein and probed with antibodies that recognize human Mcl-1 and Flag. Two separate clones that stably express human Mcl-1 were identified and termed H1299-Mcl-1-11 and -13. (B) The control and Mcl-1 stable H1299 cells were treated with 200 nM flavopiridol for 48, 72, and 96 hours, and apoptosis was measured by incorporation of Apo-BrdUrd.

FIG. 5 NIH3T3 lines, constitutively expressing human Mcl-1, are resistant to flavopiridol-induced apoptosis. In (A), NIH3T3 cells, stably expressing human Mcl-1, and control cells were treated with 200 nM flavopiridol and harvested at 48 and 72 hours for Western analysis of Mcl-1. No reductions in Mcl-1 were seen in the stably transfected cell line. Endogenous murine Mcl-1 protein was undetectable in the wild-type NIH3T3 cells. (B) Flavopiridol reduces the levels of endogenous Mcl-1 mRNA in wild-type NIH3T3 cells. At the times indicated, total RNA was collected and probed for Mcl-1 and glyceraldehydephosphate dehydrogenase. (C) The wildtype and Mcl-1 stable NIH3T3 cells were treated with 200 nM flavopiridol for 48 and 72 h, and apoptosis was measured by incorporation of Apo-BrdUrd.

FIG. 6 Cells deficient in E2F1 are resistant to apoptosis induced by flavopiridol. (A) H1299 cell lines stably transfected with E2F1 RNAi vector were created and compared with control vector-derived cells. No detectable E2F1 protein was found in these cells, whereas abundant E2F1 protein was found in control vector cells. Both control H1299 and E2F1 “knockdown” H1299 cells were treated with flavopiridol for 8 hours and total protein analyzed for E2F1 and Mcl-1. (B) Control and E2F1-deleted H1299 cell lines were treated with 200 nM flavopiridol for 48 and 72 h and harvested for apoptosis measured by the percentage of cells incorporating Apo-BrdUrd. (C) Cell lines derived from E2F1 null mouse embryo fibroblasts and wild-type littermates were treated with 200 nM flavopiridol for 48 and 72 hours and subsequently harvested for apoptosis measured by the percentage of cells incorporating Apo-BrdUrd. Cells lacking E2F1 were resistant to apoptosis induced by flavopiridol compared with control wild-type cells.

FIG. 7 A mechanism of flavopiridol-induced apoptosis. Flavopiridol inhibits the kinase activity of cyclin A/cdk2 complexes that normally serve to negatively regulate E2F1 levels during S phase. The release of negative regulation of E2F1 allows for elevations in E2F1 levels that may contribute to transcriptional repression of Mcl-1 as well as activation of other E2F1- dependent apoptotic pathways. Flavopiridol may also directly target other E2F1-independent pathways that regulate Mcl-1 levels. Reductions in Mcl-1 levels change the balance of pro- and antiapoptotic Bcl-2 family members and result in apoptosis.

FIG. 8 shows the BS/U6 E2F1 RNAi vector.

BRIEF DESCRIPTION OF SEQUENCES

<110> University of South Florida
<120> An E2F1 RNA Inhibitor to Prevent Apoptosis
<130> 1372.133
<160> 4
<170> PatentIn version 3.2
<210> 1
<211> 21
<212> DNA
<213> Artificial Sequence
<220>
<223> Oligo 1a (SEQ. NO. 1) and 1b (SEQ. NO. 2)
were annealed and ligated into the ApaI (made
blunt with T4 DNA polymerase) and HindIII sites
of the BS/U6 RNAi plasmid to form an intermediate
vector.
<400> 1
gggggagaag tcacgctatg a 21
<210> 2
<211> 24
<212> DNA
<213> Artificial Sequence
<220>
<223> Oligo 1a (SEQ. NO. 1) and 1b (SEQ. NO. 2)
were annealed and ligated into the Apa1 (made
blunt with T4 DNA polymerase) and HindIII sites
of the BS/U6 RNAi plasmid to form an intermediate
vector.
<400> 2
gcttcatagc gtgacttctc cccc 24
<210> 3
<211> 30
<212> DNA
<213> Artificial Sequence
<220>
<223> E2F1 RNAi oligo 1c (SEQ. NO. 3) and 1d (SEQ.
NO.4) were annealed and ligated into the HindIII
and EcoRI sites of the intermediate vector.
<400> 3
gcttcatagc gtgacttctc cccctttttg 30
<210> 4
<211> 30
<212> DNA
<213> Artificial Sequence
<220>
<223> E2F1 RNAi oligo 1c (SEQ. NO. 3) and 1d (SEQ.
NO.4) were annealed and ligated into the HindIII
and EcoRI sites of the intermediate vector.
<400> 4
attcaaaaag ggggagaagt cacgctatga 30

### DETAILED DESCRIPTION OF THE PREFERRED EMBODIMENT

The H1299 non-small cell lung cancer line are grown in DMEM supplemented with 2 mM L-glutamine (Life Technologies, Inc.), 5% fetal bovine serum (Hyclone), and 1% penicillin/streptomycin (Life Technologies, Inc.). NIH3T3 cells constitutively expressing human Mcl-1 were created by transfecting cells with pcDNA-3-Mcl-1 and selecting for transformants in the presence of 400 μg/ml G418. Mouse embryo fibroblasts from E2F1-null animals were grown in DMEM with 15% fetal bovine serum and 1% penicillin/streptomycin. A 50-mM stock of flavopiridol was maintained in DMSO and stored at −70° C. The drug was diluted directly into the medium to indicated concentrations followed by incubation at 37° C. Untreated cells and cells treated with DMSO alone behaved identically.

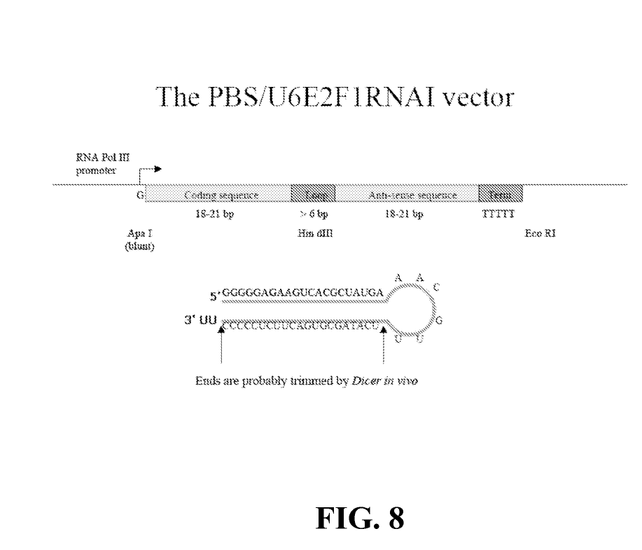

A 21-nucleotide region within codons 123-130 of human E2F1 was targeted to generate the BS/U6 E2F1 RNAi plasmid (shown in FIG. 8). This sequence was chosen because it began with a run of Gs, it was more than 100 bp within the coding region of E2F1, it was conserved in both mouse and human, and it did not significantly match any other genes in a Basic Local Alignment Search Tool search. In the first step of the construction, E2F1 RNAi oligo 1a (SEQ. NO. 1) and 1b (SEQ. NO. 2) were annealed and ligated into the Apa I (made blunt with T4 DNA polymerase) and Hin dIII sites of BS/U6. In the second step, E2F1 RNAi oligo 1c (SEQ. NO. 3) and 1d (SEQ. NO. 4) were annealed and ligated into the Hin dIII and Eco RI sites of the intermediate vector. The final vector expresses a single-stranded RNA from the U6 promoter, which is predicted to form a 21-bp double-stranded stem, a 6-bp loop, and, at the 3′-end, a short stretch of thymidine residues corresponding to the termination sequence of RNA polymerase III. The sequence of oligo 1a (SEQ. NO. 1) is 5′-GGGGGAGAAGTCACGCTATGA-3′, oligo 1b (SEQ. NO. 2) is 5′-GCTTCATAGCGTGACTTCTCCCCC-3′, oligo 1c (SEQ. NO. 3) is 5′-GCTTCATAGCGTGACTTCTCCCCCTTTTTG-3′, and oligo 1d (SEQ. NO. 4) is 5′-ATTCAAAAAGGGGGAGAAGTCACGCTATGA-3′. To determine whether the E2F1 RNAi was functional; it was cotransfected together with a pcDNA3-E2F1 expression vector. The BS/U6 E2F1 RNAi vector abolished expression of E2F1 from the vector as measured by both E2F1 western and by a luciferase assay, which measured E2F1 transcriptional activation (data not shown).

To generate E2F1 knockdown cell lines H1299 cells were transfected by the calcium phosphate method either with empty BS/U6 RNAi vector or with BS/U6-E2F1 RNAi vector together with pcDNA-3, which encodes a neomycin-selectable marker. Forty-eight hours after transfection, cells were split and cultured in medium containing 400 μg/ml G418. Colonies that emerged from G418 selection were propagated and screened for E2F1 expression by Western blot. Several lines were isolated that had reduced E2F1 expression; the most efficient knockdown cell line, H1299-E2F1RNAi-16, was further characterized. A matching H1299-pBS/U6 cell line was generated in the same way.

H1299 cells lines, constitutively expressing human Mcl-1 under control of the CMV promoter, were obtained by transfecting cells with pcDNA3 or pcDNA3-Mcl-1 and selecting for transformants by growth in 400 μg/ml G418. G418-resistant lines were screened for expression of human Mcl-1. Two Mcl-1-positive cell lines emerged and were characterized. A matching G418-resistant H1299-pcDNA3 cell line was generated in the same way.

Cell lysates were normalized for total protein content (50 μg) and subjected to SDS-PAGE as described previously. Primary antibodies used in these studies consisted of E2F1 (SantaCruz Biotechnology; SC-251), Mcl-1 (Santa Cruz; SC-819), Rb (PharMingen; 14031A), Flag (Sigma; F-3615) and β-actin (Sigma; A5441). Detection of proteins was accomplished using horseradish-peroxidase-conjugated secondary antibodies and enhanced chemiluminescence (ECL) purchased through Amersham. Northern blots were performed as described previously.

Apoptosis was assayed using PharMingen “APO-BrdU” kit without modification. After treatment with flavopiridol, cells were trypsinized and resuspended in PBS. Cells were counted and 1-2×106 cells were fixed in 1% paraformaldehyde in PBS on ice, pelleted, washed twice in PBS, and fixed with ice-cold 70% ethanol overnight. The next day, cells were pelleted, resuspended, and washed with wash buffer. Pelleted cells were resuspended with reaction buffer, TdT enzyme, and bromo-dUTP for 1 hour at 37° C. Cells were subsequently rinsed with 1.0 ml of PBS and resuspended with fluorescein-labeled anti-BrdUrd in the dark for 30 min at room temperature. Propidium iodide and RNase were added, and the cells were incubated for 30 min. One×104 cells per experimental condition were analyzed for fluorescence on a Becton-Dickinson FACScan using Cell Quest software. All of the experiments measuring apoptosis were performed at least twice (unless otherwise mentioned in the text) to ensure reproducibility of results; one such experiment is demonstrated in the figures.

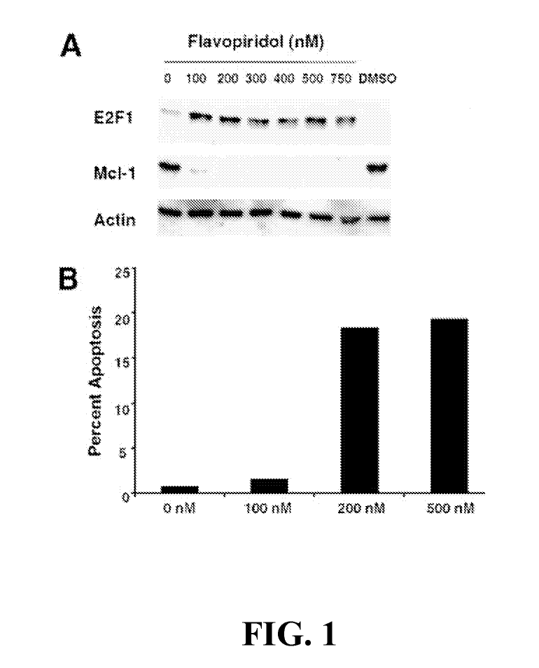

Dose-dependent apoptosis induced by flavopiridol in H1299 cells is associated with enhanced levels of E2F1 and reductions in Mcl-1. Initial experiments used the H1299 human lung carcinoma cell line that lacks a functional p16, has wild-type Rb, and lacks p53 function through a genomic deletion. These cells, therefore, are useful in delineating apoptotic pathways that do not require the action of p53. H1299 cells were treated with different doses of flavopiridol, and total protein was run on SDS-PAGE for immunoblots of E2F1 and Mcl-1. As demonstrated in FIG. 1A, untreated cells have low levels of E2F1 but easily detected Mcl-1 protein levels. After treatment with flavopiridol, E2F1 levels become markedly elevated starting at the 100-nM dose, and additional escalations in the dose of flavopiridol, above 200 nM of flavopiridol, have minimal increases in E2F1 levels. Conversely, Mcl-1 levels are significantly reduced starting at the 100-nM dose, and by 200 nM and higher doses of flavopiridol, the protein levels of Mcl-1 are undetectable. The membranes can be stripped and probed with antibody for actin to demonstrate equal protein loading. To determine the consequence of flavopiridol treatment, H1299 cells, treated in an identical manner, are assayed for apoptosis after 48 hours of treatment. As shown in FIG. 1B, minimal elevations in apoptosis, as measured by Apo-BrdUrd incorporation, are seen until a dose of 200 nM was reached. Additional increases in flavopiridol levels have no further elevations in degree of apoptosis. Taken together, these data show that apoptosis induction by flavopiridol is associated with increased E2F1 activity and reduction in Mcl-1.

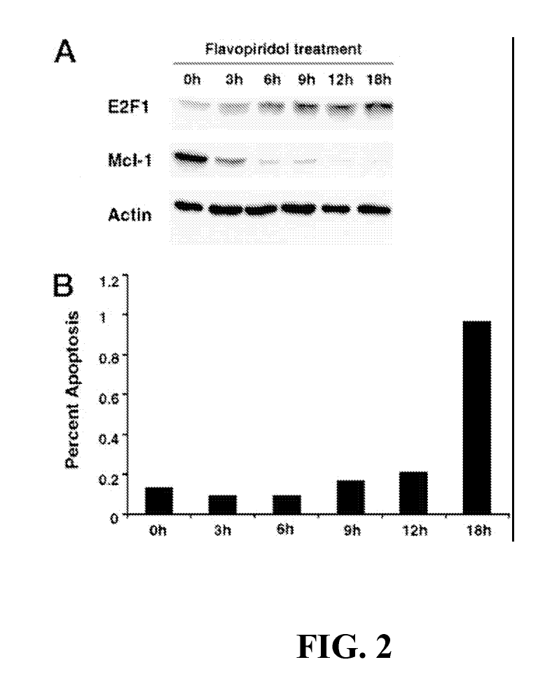

Flavopiridol leads to a time-dependent increase in E2F1 and decrease in Mcl-1 protein levels. H1299 cells are treated with a one-time dose of 200 nM flavopiridol and harvested at various times after treatment for apoptosis assays and immunoblots for E2F1 and Mcl-1 to better evaluate the linkage between the induction of E2F1 and reduction in Mcl-1 levels after flavopiridol. The Western blot shown in FIG. 2A demonstrates that increases in E2F1 protein are apparent after 3 hours of treatment and that E2F1 levels reached a plateau after 9 hours. Similarly, Mcl-1 levels began to fall after 3 hours of treatment and were maximally reduced after 12 hours. Actin levels remain unchanged, indicating equal protein loading. No changes in Bok or Bcl-2 are seen with flavopiridol treatment, which is consistent with the prior art. As shown in FIG. 2B, treatment of H1299 cells with 200 nM flavopiridol had minimal effect on apoptosis at times <18 hours, which is consistent with prior art relating to A549 lung carcinoma cells, which demonstrate apoptosis only after longer periods of treatment. These results show that elevations in E2F1 and reductions in Mcl-1 protein clearly precede the onset of apoptosis in H1299 cells and are not the consequence of apoptosis.

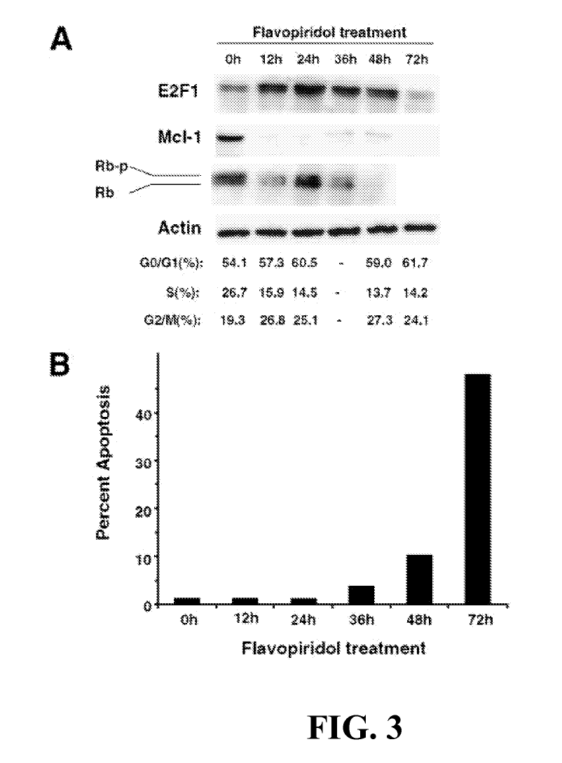

Elevations of E2F1 and reduction of Mcl-1 precede apoptosis induction by flavopiridol. As demonstrated above, the induction of apoptosis clearly occurs after the rise of E2F1 and reduction in Mcl-1 levels. An experiment identical to that shown in FIG. 2, with 200 nM flavopiridol, but with extended time-course analysis of E2F1 and Mcl-1 levels and apoptosis was done. Consistent with previous results, FIG. 3A demonstrates that after 12 hours of treatment, E2F1 levels had risen, whereas Mcl-1 levels were undetectable after 12 hours. Low levels of Mcl-1 were apparent by 36 hours, because of reduced flavopiridol levels occurring through its degradation. Similarly, E2F1 levels fell after 72 hours of treatment, again, because of reduced levels of flavopiridol. Actin levels were equal, which was consistent with equal loading of proteins on the gel. The changes in Rb phosphorylation induced by flavopiridol treatment were also examined. Flavopiridol can inhibit cdk activity, and the resulting Rb dephosphorylation may lead to enhanced Rb-E2F1binding and reduced E2F1 activity. To address this concern, Rb Western blots were performed at the indicated times after flavopiridol treatment. Although Rb protein levels drop late in the time course as cells enter apoptosis, these data demonstrate no changes in Rb phosphorylation in H1299 cells treated with flavopiridol. These results reiterate persistent Rb phosphorylation in H1299 cells overexpressing the cdk inhibitor p16. Also examined were cell cycle changes in these cells by propidium iodide staining. As a result, 1113% fewer cells underwent DNA synthesis when treated with flavopiridol, consistent with a mechanism of cdk2 and cdk4 inhibition. Apoptosis was also examined in these cells. Minimal levels of apoptosis were found after 24 hours of treatment, but with longer periods of treatment with 200 nM of flavopiridol, apoptosis was evident at 36 hours, and larger degrees of apoptosis were seen after 48 and 72 hours (FIG. 3B). This is consistent with the prior art relating to A549 lung carcinoma cells, which similarly required 48-72 hours to see the maximal effect of flavopiridol. These findings further suggest that the apoptosis induced by flavopiridol may proceed through the induction of E2F1 and subsequent repression of Mcl-1. Furthermore, these experiments confirm that elevations in E2F1 and reductions in Mcl-1 levels with flavopiridol treatment clearly precede the onset of apoptosis.

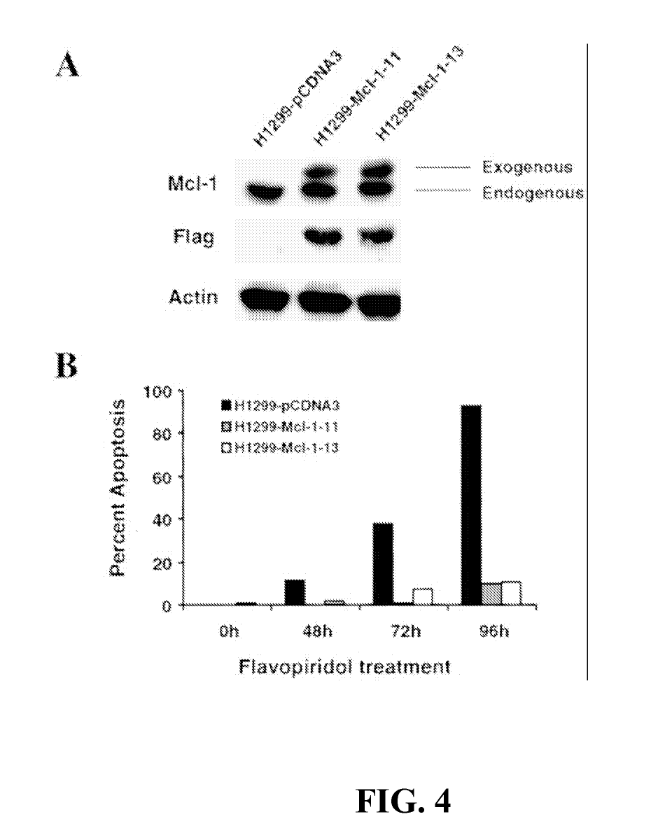

Cells that constitutively express Mcl-1 are resistant to apoptosis induced by flavopiridol. A study of the effect of constitutive Mcl-1 expression on flavopiridol-induced apoptosis was performed to determine whether Mcl-1 down-regulation is a necessary event for apoptosis induced by flavopiridol. To this end, H1299 cells were created and stably transfected with human Mcl-1 driven from the exogenous CMV promoter and isolated two separate clones that express a flag-tagged human Mcl-1 protein. As shown in FIG. 4A, these two cell lines, labeled H1299-Mcl-1-11 and H1299-Mcl-1-13, expressed a slower migrating Mcl-1 protein consistent with the addition of the flag motif. These cells also contained a flag-tagged protein running at the same mobility as Mcl-1. Control cells consisted of pcDNA3 stably transfected H1299 cells. These cells were treated with 200 nM flavopiridol and harvested after 48, 72, and 96 hours for apoptosis assays. As shown in FIG. 4B, both of the stably transfected Mcl-1 clones are resistant to apoptosis induced by flavopiridol compared with the control cells. This shows that down-regulation of Mcl-1 is required for flavopiridol-induced cell death in H1299 lung cancer cells.

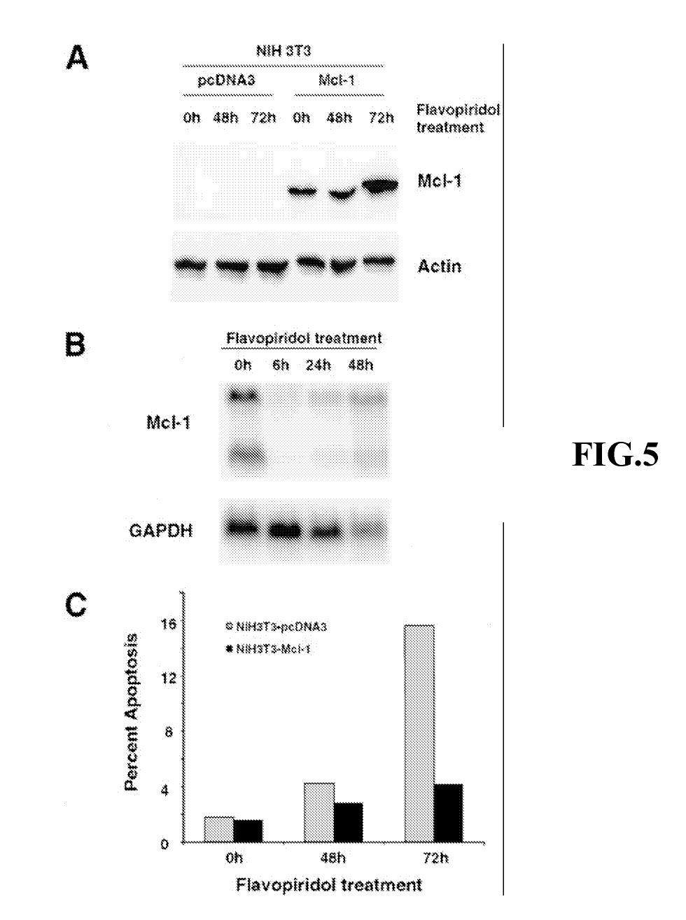

The necessity of Mcl-1 reduction in NIH3T3 fibroblasts was also determined. Previous work with these cells demonstrated that CMV-driven Mcl-1 is not repressed by E2F1 and that these cells are resistant to apoptosis induced by E2F1. These cells were tested for their ability to undergo apoptosis after treatment with flavopiridol. NIH3T3 cells, stably transfected with pcDNA3-Mcl-1, were generated (as described previously) in which human Mcl-1 expression is driven from the exogenous CMV promoter. Immunoblots of parallel treated NIH3T3 and cells expressing Mcl-1 are shown in FIG. 5A. No Mcl-1 is detectable in these murine cells because the antibody recognizes only human Mcl-1. The stably transfected cells have easily detectable human Mcl-1 protein, and no changes are seen in the levels of Mcl-1 despite 72 hours of treatment with flavopiridol. This result demonstrates a brisk reduction in Mcl-1 protein levels with flavopiridol treatment in H1299 lung carcinoma cells. To demonstrate that flavopiridol reduces Mcl-1 expression in the 3T3 cells, cells were treated with flavopiridol for different times and total RNA collected. FIG. 5B demonstrates that flavopiridol reduces the levels of endogenous Mcl-1 in wild-type 3T3 cells within 6 hours of treatment. FIG. 5C demonstrates that the wild-type NIH3T3 cell line, transfected with pcDNA3 alone, undergoes apoptosis starting after 48 hours but is more pronounced after 72 hours of treatment. Conversely, the cell lines stably expressing Mcl-1 have minimal increases in apoptosis despite 72 hours of treatment. These results demonstrate that preventing the ability of flavopiridol to deplete Mcl-1 expression leads to the inhibition of flavopiridol-mediated cell death, thereby suggesting that the down-regulation of Mcl-1 by flavopiridol may be a critical, or perhaps necessary, event in flavopiridol-induced apoptosis. Furthermore, these data are consistent with results suggesting that constitutive Mcl-1 expression prevents apoptosis induced by E2F1 overexpression.

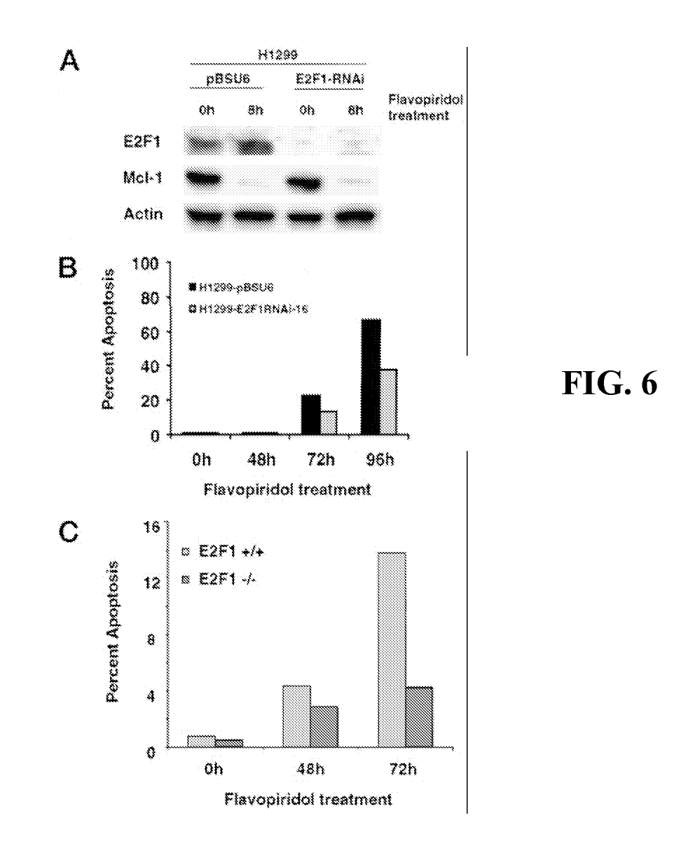

Cells lacking E2F1 are less sensitive to apoptosis induced by flavopiridol. Resulting data demonstrate that apoptosis induced by flavopiridol results in a parallel rise in E2F1 levels and reduction in Mcl-1 levels, and cell lines that constitutively express Mcl-1 (and are resistant to repression by E2F1) are resistant to apoptosis induced by flavopiridol. To directly demonstrate that an elevation in E2F1 protein level is a necessary event for apoptosis resulting from flavopiridol treatment, the effect of flavopiridol on cells lacking E2F1 was studied. For these experiments, an H1299 cell line was created that does not express detectable E2F1 by virtue of expression of a small hairpin RNA inhibitor, as discussed supra. FIG. 6A demonstrates that the E2F1 protein is absent in these cells, and neither could Northern analysis detect E2F1 mRNA in these cells (data not shown). However, when these cells were treated with flavopiridol, Mcl-1 protein levels decreased in a manner identical to that of the control cells. This suggests that flavopiridol can reduce Mcl-1 protein and mRNA levels through an E2F1-independent mechanism. When assayed for apoptosis, H1299 cells lacking E2F1 protein were less sensitive to flavopiridol-induced cell death compared with H1299 cells transfected with an empty RNAi vector. This shows that E2F1 is required for the full apoptosis that is elicited by flavopiridol treatment. Cell lines generated from mouse embryo fibroblasts acquired from animals lacking the E2F1 gene addition to the H1299 E2F1-deleted cell lines. Control cells consisted of cell lines from mouse embryo fibroblasts from littermates of the E2F1 knockout mice that retained both copies of the E2F1 gene. Cells were treated with 200 nM flavopiridol and harvested after 48 and 72 hours for assays of apoptosis. As shown in FIG. 6C, cells lacking E2F1 are less sensitive to flavopiridol-induced cell death after 72 hours of treatment, consistent with a role of E2F1 in flavopiridol induced apoptosis. These important results with cells lacking E2F1, coupled with data showing elevations in E2F1 with flavopiridol treatment, provides genetic proof for a role of E2F1 in mediating the apoptotic function of flavopiridol.

It has been known for some time that E2F1, the first member of the E2F family identified, can induce not only S-phase entry but also apoptosis. The apoptotic function of E2F1 is classically p53-dependent, because mouse embryo fibroblasts from homozygous p53 knockout mice showed marked reduction in the amount of cell death induced by E2F1 overexpression. Although the classic pathway for E2F1-induced apoptosis occurs through the ARF-Mdm2-p53 pathway, the prior art has demonstrated that E2F1 can induce cell death in cells deficient in either ARF or p53. Another p53 family member, p73, is directly activated by E2F1 through its promoter, and an additional study demonstrated that Apaf-1 is a direct transcriptional target of E2F1. In addition, and in some contexts, only the DNA-binding domain of E2F1 is required for the induction of apoptosis, and mutations in the transactivation domain can still allow for E2F1 to induce death.

The present invention characterizes a novel mechanism of E2F1-induced apoptosis. E2F1 was found to directly bind the promoter of Mcl-1 in vivo and repress its activation. The repression of Mcl-1 by E2F1 preceded apoptosis, and cells that constitutively expressed Mcl-1 were resistant to E2F1-induced cell death. The present invention demonstrates that one mechanism of apoptosis induced by flavopiridol is through an E2F1-dependent pathway. Although originally designed to be cytostatic agents that arrested cell cycle progression, agents that target and inhibit cdk activity have also been linked to inducing apoptosis in different cell types. One suggested mechanism of such action is through the inhibition of cyclin A/cdk2 activity, which normally serves to down-regulate E2F activity through phosphorylation of E2F1:DP1 dimers. The present invention discloses a method for the selective killing of tumor cells when treated with short peptides that block the interaction between cyclin A/cdk2 and E2F1. Therefore, tumors that have deregulated E2F1 activity are sensitized to undergo apoptosis when cyclin A/cdk2 activity is inhibited. Given the ability of flavopiridol to inhibit cdk2 activity with IC 5 0 of 0.1 μM, flavopiridol promotes cell death through the stabilization of E2F1. This hypothesis was coupled with observations that Mcl-1 is selectively down-regulated by flavopiridol and the fact E2F1 can directly repress Mcl-1 and induce apoptosis.

Flavopiridol and UCN-01, both small molecule inhibitors of cdks, can downregulate Mcl-1 as well as BAG-1 and XIAP. One notable difference is that flavopiridol-induced reductions in Mcl-1 and XIAP is caspase-independent, whereas UCN-01 reduces Mcl-1 and XIAP levels in a caspase-dependent manner. The activation of E2F1 by flavopiridol is unlikely to require caspase function. In addition, it has been suggested that cdk inhibition may not be the mechanism of cell death, given the nonproliferative behavior of the B-chronic lymphocytic leukemia cells. However, one study examined cell cycle proteins in peripheral blood lymphocytes from patients with B-chronic lymphocytic leukemia and found expression of cdk2, albeit at lower levels compared with nonneoplastic lymphoid tissue. This may suggest that, although cells are not cycling, they nonetheless have cdk2 activity, which, in conjunction with cyclin A, can negatively regulate E2F1 activity and prevent apoptosis induced by E2F1. A similar result of flavopiridol-inducing cell death in noncycling A549 lung carcinoma cells was seen, and one study suggested that the IC 50 of flavopiridol was significantly higher in the arrested cells. This result is consistent with flavopiridol acting to inhibit cdk2/cyclin A-mediated down-regulation of E2F1 in that cycling cells have higher levels of E2F1 and would be more sensitive to flavopiridol. Flavopiridol induces apoptosis through a p53-independent mechanism which is consistent with the present invention demonstrating that repression of Mcl-1 is one mechanism of flavopiridol-induced death.

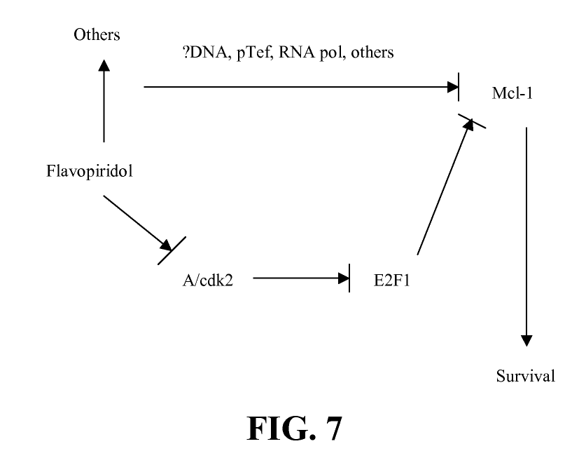

Because E2F1 knockout cells are not completely resistant to apoptosis induced by flavopiridol, other mechanisms may also contribute (FIG. 7). Indeed, results with the H1299 cell line (with absent E2F1 similarly demonstrating a reduction in Mcl-1 with flavopiridol treatment) argue against a mechanism of Mcl-1 repression mediated solely by E2F1. For example, other members of the E2F family may be involved, because both E2F2 and E2F3A are targets of cyclin A/cdk2. Alternatively, Mcl-1 repression by flavopiridol may be completely independent of E2F function altogether. As an illustration, one mechanism of action of flavopiridol has been suggested through genomic scale measurement of gene expression using DNA microarray technology. The present invention demonstrates that flavopiridol affects gene expression in a manner analogous to other transcriptional inhibitors such as actinomycin D. One potential mechanism of such widespread changes in gene expression is through the recent finding that flavopiridol inhibits the activity of p-TEGb, a transcriptional elongation factor. This is consistent with other reports that the inhibition of cdk2 activity can affect CMV replication, as well as with reports that roscovitine, a potent inhibitor of cdk2, cdk5, and cdk7, can inhibit RNA synthesis by the inhibition of phosphorylation of RNA polymerase II. Nonetheless, E2F1 contributes significantly to the apoptotic function of flavopiridol. Furthermore, both the H1299 and the NIH3T3 cell lines, which constitutively express Mcl-1 under the direction of the CMV promoter, do not demonstrate reductions in Mcl-1 levels, and these cells are resistant to apoptosis induced by flavopiridol.

In addition to being regulated by E2F1, the Mcl-1 gene is also regulated by survival signals initiated from tyrosine kinase and cytokine signaling pathways. The prior art has demonstrated that Stat3, a member of the STATs family of transcription factors, can have an antiapoptotic effect through the up-regulation of Mcl-1. In addition, phosphatidylinositol 3′ kinase and Akt can upregulate Mcl-1 through the action of CREB. Finally, the promoter of Mcl-1 contains AP-1 binding sites, and studies have reported the ability of inhibitors of mitogen-activated protein kinase signaling to cause reductions in Mcl-1 expression. Therefore, expression of Mcl-1 is under tight control from many survival signals implicated in the formation of human cancers. One hypothesis relevant for the treatment of human cancers with flavopiridol, and possibly other cdk2 inhibitors, is that survival signaling pathways regulated by tyrosine kinase signaling, such as Stat3 and Akt, may modulate the level of cell death induced by flavopiridol by regulating the Mcl-1 gene. Therefore, tumors that prevent the down-regulation of Mcl-1 in response to elevated E2F1 levels may be resistant to cell death induced by flavopiridol. A colon carcinoma cell line that is resistant to flavopiridol has been generated but the mechanism of resistance is currently unclear. Understanding this mechanism may allow the development of assays on tumor specimens that can predict which patients will respond to flavopiridol, as well as other novel inhibitors of cdk2 activity.

The disclosure of all publications cited above are expressly incorporated herein by reference, each in its entirety, to the same extent as if each were incorporated by reference individually.

It will be seen that the advantages set forth above, and those made apparent from the foregoing description, are efficiently attained and since certain changes may be made in the above construction without departing from the scope of the invention, it is intended that all matters contained in the foregoing description or shown in the accompanying drawings shall be interpreted as illustrative and not in a limiting sense.

It is also to be understood that the following claims are intended to cover all of the generic and specific features of the invention herein described, and all statements of the scope of the invention which, as a matter of language, might be said to fall therebetween. Now that the invention has been described,

What is claimed is:

1. A method of inducing apoptosis in a target cell population comprising the steps of:
introducing flavipiridol to the target cell population; and
introducing one or more compounds that increase the expression of E2F1.

2. The method of claim 1 further comprising the step of introducing one or more compounds that decrease the expression of Mcl-1.

3. A method of inducing apoptosis in a target cell population comprising the steps of:
introducing flavipiridol to the target cell population; and
introducing one or more compounds that decrease the expression of Mcl-1.

4. The method of claim 3 further comprising the step of introducing one or more compounds that increase the expression of E2F1.


SEQUENCE LISTING (4 sequences)

SEQ ID NO: 1 - DNA, 21 bp, Artificial Sequence
  Oligo 1a (SEQ. NO. 1) and 1b (SEQ. NO. 2) were annealed and ligated into the ApaI (made blunt with T4 DNA polymerase) and HindIII sites of the BS/U6 RNAi plasmid to form an intermediate vector.
gggggagaagtcacgctatga

SEQ ID NO: 2 - DNA, 24 bp, Artificial Sequence
  Oligo 1a (SEQ. NO. 1) and 1b (SEQ. NO. 2) were annealed and ligated into the ApaI (made blunt with T4 DNA polymerase) and HindIII sites of the BS/U6 RNAi plasmid to form an intermediate vector.
gcttcatagcgtgacttctccccc

SEQ ID NO: 3 - DNA, 30 bp, Artificial Sequence
  E2F1 RNAi oligo 1c (SEQ. NO. 3) and 1d (SEQ. NO.4) were annealed and ligated into the HindIII and EcoRI sites of the intermediate vector.
gcttcatagcgtgacttctccccctttttg

SEQ ID NO: 4 - DNA, 30 bp, Artificial Sequence
  E2F1 RNAi oligo 1c (SEQ. NO. 3) and 1d (SEQ. NO.4) were annealed and ligated into the HindIII and EcoRI sites of the intermediate vector.
attcaaaaagggggagaagtcacgctatga